(19) United States
(12) Patent Application Publication
(10) Pub. No.: US 2006/0079808 A1
(43) Pub. Date: Apr. 13, 2006

(54) Blood collecting syringe with retractable needle

(76) Inventor: Edward F. Allard, Springfield, VA (US)

Correspondence Address: Edward F. Allard, 7830 Greeley Blvd., Springfield, VA, 22152, US

(21) Appl. No.: 10/962,132

(22) Filed: Oct. 12, 2004

(51) Int. Cl. A61B 5/00

(52) U.S. Cl. 600/576; 604/110

(57) ABSTRACT

A blood-taking syringe having a retractable needle, releasable upon demand, having a needle assembly, under tension from a compressed spring, with a needle holding head supporting a double ended needle axially aligned with the central axis of the syringe, which is held in equilibrium by a releasable annular ring interposed within a slot on the inside of the syringe housing. A release pin extends through the side of the syringe at the point where the annular ring is located and is disposed such that upon depressing the pin the ring is dislodged from the slot in the syringe housing, releasing the needle assembly to be forced into the syringe for safe storage.

### BACKGROUND OF THE INVENTION

1. Field of the Invention

This invention relates to blood-taking syringes, and more particularly to a syringe having a needle assembly which may, upon demand, be retracted into the housing of the syringe, in such a way that the needle becomes inaccessible, is rendered non-operational and the housing becomes a safe storage container for the spent needle and for any residual fluids.

2. Description of the Prior Art

The spread of diseases from the inadvertent exposure to contaminated needles has become a major concern throughout the health industry. Numerous attempts have been made to reduce the likelihood of the user being exposed to needle pricks and residual body fluids, by designing various schemes to cap the spent needle or to retract the needle into the cavity of the syringe, but none of the approaches have proven to be entirely satisfactory in providing an easily actuated mechanism for retracting and safely storing the needle in a fail-safe container that can then be safely handled without fear of being exposed to the spent needle and collected fluids. The instant technique and apparatus for collecting blood and safely disposing of the spent needle fully meets the existent needs of the health care worker and effectively overcomes all the safety concerns of the prior art devices.

### SUMMARY OF THE INVENTION

The general purpose of this invention is to provide an operationally simple, easy, safe and effective way to retract the needle assembly of a blood taking device, upon demand, into the syringe housing, to prevent the device from being reused, and then to seal the housing, rendering it a safe storage container for the needle assembly.

### BRIEF DESCRIPTION OF DRAWINGS

FIG. 1 shows a cut-away view of a blood-taking device as envisioned by this invention.

FIG. 2 shows the needle assembly of the blood-taking device in a retracted state.

### DESCRIPTION OF THE PREFERRED EMBODIMENTS

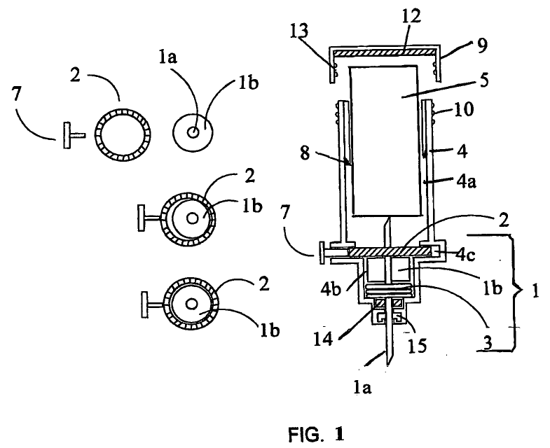

This invention will be best understood by referring to the drawings. Looking now at the blood taking syringe of FIG. 1, note the syringe housing (4) having first and second chambers, (4a) and 4(b), respectively, with the first chamber (4a) adapted to receive a blood taking vacuum vial (5) and with the second chamber (4b) designed to hold a spring loaded, double ended needle (1a) within a needle holder head (1b) of the hypodermic needle head assembly (1). The double ended needle (1a) is mounted within a needle head assembly (1) and extends axially therethrough, allowing the distal end of the needle to be inserted into a vein of a blood giver while the other end punctures one end of a vacuum vial (5) upon inserting the vial into chamber (4a) and inserting moderate pressure thereto. As is well known in the art, the vacuum within the vacuum vial (5) then draws blood from the vein of the patient and fills the vial accordingly. Any desired number of vials of blood may be drawn from the patient before initiating the withdrawal of the needle from the patient and releasing the needle head assembly (1) for storage within chamber (4a) of syringe housing (4). A compressed spring (3) tends to force the needle assembly (1) into the inside of the upper chamber (4a) of the syringe housing (4), however, spring (3) is prevented from driving the head assembly (1) into chamber (4a) by an annular hold-down ring (2), which can be fabricated of hard plastic or vinyl or any other material strong enough to withstand the force of the compressed spring (3) and apply a counter force to the needle assembly (1) and hold the needle assembly in a state of equilibrium from the force of the compressed spring (3). Retraction of the needle head assembly (1) may be effected, upon demand, by depressing the release pin (7), whereupon the end of pin (7) is forced against the edge of the hold-down ring (2), forcing the ring into the center of the syringe housing (4). Note that a syringe projection, having a slot (4c) therein, extends entirely around the periphery of the syringe housing (4) at the intersection of the syringe housing chambers (4a) and (4b) and that the slot is of such a width and depth that the thickness of the annular ring (2) readily fits therein. The annular ring (2) has a hole in the center thereof sufficiently large to allow the needle assembly to unobstructively pass therethrough upon alignment of the axis of annular ring (2) and the central axis of the syringe housing (4), however, when the was of the annular ring (2) and the axis of the syringe (4) are not coincident, the annular ring, being off-center, functions to restrain the needle assembly (1) by stopping the upward movement of the needle assembly (1) with one edge of the annular ring (2) being in contact with needle holder (1b). The centerline axis of the annular ring is normally offset from the central axis of the syringe housing (4), thusly restraining the needle assembly (1) until activation of the release pin (7). Prior to activating the release pin (7), the safety cap (9) is attached to the syringe housing, which is here shown as a screw-on type, but may be of any design that would securely seal the cap when applied to the container. The safety cap (9) is shown as twisted onto the guide (10) and may be similar to a push down, commercial safety cap used on safe liquid medicine bottles for protecting children. The safety cap seal (12) seals the top of chamber (4a) upon placing the safety cap (9) on the syringe housing and accordingly seals in any fluid remaining in chamber (4a) upon removal of the vacuum vial (5), as well as sealing chamber (4a) for accepting the needle assembly upon release of the needle restraining means. Safety cap (11) is only one example, among many, that may be used to effect the sealing of the needle within the syringe. Broken rings (13) on the cap screw are mated with ring guide (10) on the housing. When the cap is screwed onto the housing, it seals that end of the housing, eliminating the possibility of the inadvertent removal of the cap and spilling contaminated fluid. Upon activation of the needle release means the compressed spring (3) forces the needle head assembly (1) into chamber (4a) of the syringe for storage. A push-in motion applied to the push tab (7) releases the holding ring (2) which can be performed easily with one hand. The spring then would be free to expand and force the needle head assembly into the container holder. The inner cylinder (8) provides a guide for vacuum vial (5), but is not an essential part of the syringe. If desired, the inner cylinder (8) may be molded into the syringe upon manufacture or may be a simple sleeve inserted within the syringe housing. Once the needle assembly is retracted into the syringe housing chamber (4a), it cannot be reused. When the cap is screwed onto the housing, it seals that end of the housing, eliminating the possibility of the inadvertent removal of the cap and spilling contaminated fluid. Seal (14) is a self-sealing seal that immediately seals the hole left by the needle after retraction. This self-sealing material seals the lower end of the syringe housing, which prevents any inside contaminated fluid from leaking to the outside and prevents the retracted needle from protruding back through the hole left by retraction of the needle (1a). Cavity (15) captures any droplets of blood that might be wiped off the needle (1a) by the seal (14) during retraction, however, an additional cap (not shown), similar to that of cap (9), may be used on the outermost end of the second chamber (4b) of the syringe housing (4), in the event that one desires to be assured that the spent needle and other body fluids are absolutely encapsulated within the housing upon retraction of the needle assembly. The two end caps (9) may be attached to the syringe upon manufacture and removed prior to use, in order to protect the needle from any potential environmental contamination.

Upon attaching safety caps (9) on the outermost ends of chambers (4a) and (4b) and upon retraction of the needle assembly (1) into chamber (4a) of the syringe housing (4), the needle head assembly and any contaminated fluids within the housing of the syringe (4) are encapsulated within the syringe housing (4), thereby rendering the syringe housing (4) a safe container for users and handlers who may come in contact with the used syringe.

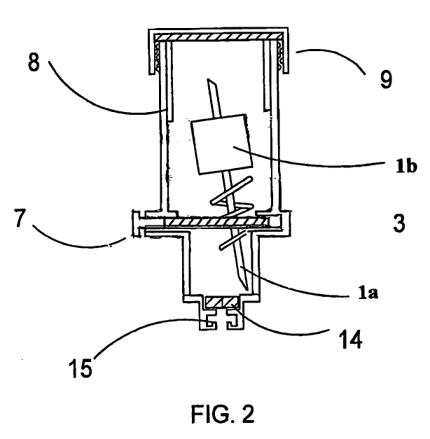

FIG. 2 shows the blood-taking device of FIG. 1 in its retracted state. Safety cap (9) has been twisted onto the top of the container, effectively sealing and securing the top end of the syringe. Push tab (7) has been activated, forcing the annular ring (2) into central alignment with the axis of the of the syringe housing and thereby releasing the spring biased needle assembly (1) and forcing the needle assembly (1) into chamber (4a) of the syringe housing (4). The needle assembly (1) and spring (3) have been retracted. The needle seal (14) has automatically closed, sealing the bottom of the container. The container cannot be used again, thus the needle is inaccessible and the container is completely sealed, thereby rendering the used syringe (4) a safe container for the spent needle and fluids.

While this invention has been described in terms of a preferred embodiment, those skilled in the art will recognize that the invention can be practiced with modification within the spirit and scope of the appended claims.

Having thus described my invention, what I claim as new and desire to secure by Letters Patent is as follows:

1. A blood taking syringe comprising:
A syringe housing having a first chamber adapted to accept a removable blood-collecting vacuum vial and a second chamber adapted to hold a releasably mounted needle assembly under tension with an outwardly extending protrusion on the syringe housing at the intersection of the first and second chambers of the syringe housing, with said protrusion having an inwardly open slot extending entirely around the periphery of the syringe housing;
A needle assembly, including a cylindrically shaped needle retainer, having first and second ends, with a double-ended hypodermic needle extending through the center thereof, with said needle and needle retainer being axially aligned with the central axis of the syringe housing;
A compressable spring, normally held in a compressed mode, seated in the second chamber of the syringe housing for applying pressure to the needle retainer along the central axis of the syringe housing;
A needle assembly restraining means for applying a restraining force to the needle retainer to counter the force of the compressed spring and for maintaining said needle assembly in a state of equilibrium, wherein the needle assembly restraining means consists of an annular ring, with the center hole being sufficiently large to allow the needle assembly to pass unobstructively therethrough, upon releasing the restraining force, with the centerline axis of the ring normally being off-set from the central axis of the syringe housing and needle assembly while in a restraining mode, during which one edge of the annular ring intrudes into the slot in the protrusion of the syringe housing to hold the needle assembly in a state of equilibrium;
Manually responsive pressure means for disengaging the needle assembly restraining means to allow the compressed spring to force the needle assembly into the first chamber of the syringe housing.
Safety cap means for stopping the retracting needle assembly and for sealing the open end of the first syringe chamber prior to release of the needle restraining means.

2. The blood-taking syringe of claim 1, wherein at least one hole extends through the wall of the housing, at the site of the slot and the syringe protrusion, for receiving at least one manually activated push-in tab, which upon activation, forces the portion of the annular ring within the slot in the syringe housing, into the syringe housing, and upon alignment of the centerline axis of the annular ring and the central axis of the syringe housing the needle assembly is released and forced into the first chamber of the syringe housing.

3. The blood-taking syringe of claim 2, wherein the second chamber of the syringe housing is further adapted to seat a self-sealing seal through which the distal end of the double ended needle extends, such that after drawing blood and retraction of the needle and needle assembly into the first chamber of the syringe housing, the distal end of the syringe is sealed against the leakage of any fluid remaining within the body of the syringe.

4. The blood-taking syringe of claim 3, further having a fluid collecting cavity located below the self-sealing seal, in the distal end of the syringe, which collects any residual fluid wiped from the needle during the retraction of the needle through the self-sealing seal into the first chamber of the syringe housing, thereby preventing any residual fluid from leaking to the outside of the housing.

5. The blood-taking syringe of claim 1, further having an additional safety cap means for sealing the second chamber of the syringe housing from the outside environment prior to use of the syringe and for resealing same upon use and retraction of the needle assembly.
